(19) United States
(12) Patent Application Publication
(10) Pub. No.: US 2015/0289895 A1
(43) Pub. Date: Oct. 15, 2015

(54) FLUID EJECTION DEVICE AND FLUID EJECTON METHOD

(71) Applicant: Seiko Epson Corporation, Tokyo (JP)

(72) Inventors: Masaki GOMI, Hino-shi (JP); Kazuaki UCHIDA, Fujimi-machi (JP)

(21) Appl. No.: 14/682,923

(22) Filed: Apr. 9, 2015

(30) Foreign Application Priority Data
Apr. 10, 2014 (JP) 2014-080823

(51) Int. Cl. A61B 17/3203; A61M 3/02; A61M 39/22; A61B 19/00

(52) U.S. Cl. CPC A61B 17/3203; A61B 19/46; A61M 3/02; A61M 39/22; A61B 2019/464; A61B 2017/00017

(57) ABSTRACT

A fluid ejection device includes a fluid ejection unit that ejects fluid and an ejection control unit that controls ejection of the fluid. A fluid container accommodates the fluid supplied to the fluid ejection unit. A connection channel connects the fluid ejection unit and the fluid container. A volume change unit controls opening and closing of the connection channel and adjusts an inner volume of the fluid container. The fluid ejection device operates in a first ejection mode in which, when the connection channel is opened, the volume change unit reduces the inner volume, and the ejection control unit ejects fluid from the fluid ejection unit, and after the ejection of the fluid, the connection channel is closed. In a second ejection mode, after the first ejection mode, when the connection channel is opened, the ejection control unit allows the ejection of fluid from the fluid ejection unit.

This application claims the benefit of Japanese Patent Application No. 2014-080823 filed, on Apr. 10, 2014. The content of the aforementioned application is incorporated herein by reference in its entirety.

### BACKGROUND

1. Technical Field

The present invention relates to a fluid ejection device and a fluid ejection method.

2. Related Art

A fluid ejection device for medical purposes that can incise and excise living tissue by ejecting a fluid has been developed.

JP-A-2013-213422 is an example of the related art.

In the fluid ejection device, in a case where a certain amount of time is required so as to increase pressure to an amount of pressure at which a fluid can be ejected, a user waits idly, and has difficulty in efficiently expediting an operation or the like. For this reason, it is necessary to increase a fluid pressure in the fluid ejection device to a predetermined pressure in advance.

When the fluid pressure is increased to the predetermined pressure, an inner pressure of the fluid ejection device is detected by a pressure sensor or the like. At this time, it is desirable to use a highly reliable pressure sensor or a highly accurate detectable pressure sensor. However, it may not be possible to use such an expensive pressure sensor in advancing to reduce the manufacturing costs of the fluid ejection device. Accordingly, it is desirable to reduce an amount of time taken from a demand for the ejection of the fluid to the ejection of the fluid via an inexpensive technique.

### SUMMARY

An advantage of some aspects of the invention is to inexpensively reduce an amount of time taken to eject a fluid.

A fluid ejection device according to an aspect of the invention includes: a fluid ejection unit that ejects a fluid; an ejection control unit that receives a fluid ejection command input, and controls the ejection of the fluid from the fluid ejection unit; a fluid container that accommodates the fluid to be supplied to the fluid ejection unit; a connection channel that connects the fluid ejection unit and the fluid container, and acts as a channel through which the fluid flows; an opening and closing unit that opens and closes the connection channel; a volume change unit that controls the opening and closing unit to open and close the connection channel, and adjusts an inner volume of the fluid container; and a pressure detection unit that detects an inner pressure of the fluid container. When the fluid ejection device receives the ejection command input, the fluid ejection device is configured to operate in a first ejection mode in which, regardless of an amount of the inner pressure of the fluid container, in a state where the volume change unit controls the opening and closing unit to open the connection channel, the volume change unit reduces the inner volume, and the ejection control unit ejects the fluid from the fluid ejection unit, and after the ejection of the fluid, the volume change unit controls the opening and closing unit to close the connection channel, or in a second ejection mode in which when the inner pressure of the fluid container is higher than a predetermined pressure, and in a state where the volume change unit controls the opening and closing unit to open the connection channel, the volume change unit reduces the inner volume, and the ejection control unit ejects the fluid from the fluid ejection unit, and after the ejection of the fluid, the volume change unit controls the opening and closing unit to close the connection channel.

Other features of the invention will be made apparent by the description of this specification and the accompanying drawings.

### BRIEF DESCRIPTION OF THE DRAWINGS

The invention will be described with reference to the accompanying drawings, wherein like numbers reference like elements.

FIG. 1 is a view illustrating the configuration of a fluid ejection device as an operation scalpel according to an embodiment.

FIG. 2 is a view illustrating the configuration of the fluid ejection device configured to include two pumps.

FIG. 3 is a schematic view illustrating the configuration of the pump according to the embodiment.

FIG. 4 is a view illustrating the pump with a different configuration.

FIG. 5 is a cross-sectional view illustrating the structure of a pulsation generator according to the embodiment.

FIG. 6 is a plan view illustrating the shape of an inlet channel.

FIG. 7 is a flowchart of a fluid ejection control operation.

FIG. 8 is a first graph illustrating a change in pressure in a trial mode and a normal ejection mode.

FIG. 9 is a second graph illustrating a change in pressure in the trial mode and the normal ejection mode.

### DESCRIPTION OF EXEMPLARY EMBODIMENTS

At least the following facts are apparent from this specification and the accompanying drawings.

A fluid ejection device includes: a fluid ejection unit that ejects a fluid; an ejection control unit that receives an ejection command input, and controls the ejection of the fluid from the fluid ejection unit; a fluid container that accommodates the fluid to be supplied to the fluid ejection unit; a connection channel that connects the fluid ejection unit and the fluid container, and acts as a channel through which the fluid flows; an opening and closing unit that opens and closes the connection channel; a volume change unit that controls the opening and closing unit to open and close the connection channel, and adjusts an inner volume of the fluid container; and a pressure detection unit that detects an inner pressure of the fluid container. When the fluid ejection device receives the ejection command input, the fluid ejection device is configured to operate in a first ejection mode in which, regardless of an amount of the inner pressure of the fluid container, in a state where the volume change unit controls the opening and closing unit to open the connection channel, the volume change unit reduces the inner volume, and the ejection control unit ejects the fluid from the fluid ejection unit, and after the ejection of the fluid, the volume change unit controls the opening and closing unit to close the connection channel, or in a second ejection mode in which when the inner pressure of the fluid container is higher than a predetermined pressure, and in a state where the volume change unit controls the opening and closing unit to open the connection channel, the volume change unit reduces the inner volume, and the ejection control unit ejects the fluid from the fluid ejection unit, and after the ejection of the fluid, the volume change unit controls the opening and closing unit to close the connection channel.

In this manner, in the first ejection mode, since the fluid is ejected regardless of an amount of the inner pressure of the fluid container, and thereafter, the connection channel is closed, it is possible to increase the inner pressure of the fluid container to a certain amount of pressure. In addition, in the second ejection mode, it is possible to eject the fluid by using the pressure increased in the first ejection mode. The first ejection mode can be defined as an ejection mode that is enabled so as to increase the inner pressure of the fluid container, and the second ejection mode can be defined as a normal ejection mode that is enabled so as to eject the fluid when the inner pressure of the fluid container is increased further than the predetermined pressure. At this time, since the fluid pressure is increased by the execution of the first ejection mode, even when an inexpensive and low accuracy detectable pressure unit is adopted, it is possible to reduce an amount of time taken to eject the fluid in the second ejection mode. That is, it is possible to inexpensively reduce an amount of time taken to eject the fluid.

In the fluid ejection device, when the fluid ejection device receives the ejection command input, the fluid ejection device is preferably configured to operate in the first ejection mode in which regardless of an amount of the inner pressure of the fluid container, the volume change unit reduces the inner volume while controlling the opening and closing unit to open the connection channel, and the volume change unit controls the opening and closing unit to close the connection channel after the ejection control unit ejects the fluid from the fluid ejection unit, or in the second ejection mode in which when the inner pressure of the fluid container is in a predetermined range of pressure, the volume change unit reduces the inner volume while controlling the opening and closing unit to open the connection channel, and the volume change unit controls the opening and closing unit to close the connection channel after the ejection control unit ejects the fluid from the fluid ejection unit.

In this manner, in the first ejection mode, since the fluid is ejected regardless of an amount of the inner pressure of the fluid container, and thereafter, the connection channel is closed, it is possible to increase the inner pressure of the fluid container to a certain amount of pressure. In addition, in the second ejection mode, it is possible to eject the fluid by using the pressure increased in the first ejection mode. The first ejection mode can be defined as an ejection mode that is enabled so as to increase the inner pressure of the fluid container, and the second ejection mode can be defined as a normal ejection mode that is enabled so as to eject the fluid when the inner pressure of the fluid container is within the predetermined pressure range. At this time, since the fluid pressure is increased by the execution of the first ejection mode, even when an inexpensive and low accuracy detectable pressure unit is adopted, it is possible to reduce an amount of time taken to eject the fluid in the second ejection mode. That is, it is possible to inexpensively reduce an amount of time taken to eject the fluid.

In the fluid ejection device, preferably, the fluid container includes a syringe for accommodating the fluid and a piston, and the volume change unit changes the inner volume of the fluid container by moving the piston.

In this manner, it is possible to push the fluid out of the fluid container by changing the inner volume of the fluid container.

In the fluid ejection device, in the first ejection mode, preferably, the volume change unit moves the piston at a constant speed.

In this manner, a predetermined amount of the fluid per unit time can be sent out of the fluid container.

In the fluid ejection device, preferably, the fluid ejection unit includes a nozzle through which the fluid is ejected. In the first ejection mode, preferably, the fluid is ejected through the nozzle by a constant speed movement of the piston, and the inner pressure of the fluid container converges to a predetermined pressure.

In this manner, it is possible to increase the inner pressure of the fluid container to the predetermined pressure in the first ejection mode, and then to eject the fluid at a proper intensity in the second ejection mode.

In the fluid ejection device, preferably, the piston has a gasket at the tip thereof.

In this manner, since the gasket absorbs the inner pressure of the fluid container, it is possible to increase the inner pressure of the fluid container while preventing a rapid change in pressure.

At least the following facts are apparent from this specification and the accompanying drawings.

A fluid ejection method of a fluid ejection device includes a fluid ejection unit that ejects a fluid, an ejection control unit that controls the ejection of the fluid from the fluid ejection unit, a fluid container that accommodates the fluid to be supplied to the fluid ejection unit, a connection channel that connects the fluid ejection unit and the fluid container, and acts as a channel through which the fluid flows, an opening and closing unit that opens and closes the connection channel, a volume change unit that controls the opening and closing unit to open and close the connection channel, and adjusts an inner volume of the fluid container, and a pressure detection unit that detects an inner pressure of the fluid container, the method including: performing a first ejection operation in which when the fluid ejection device receives the ejection command input, regardless of an amount of the inner pressure of the fluid container, the volume change unit reduces the inner volume while controlling the opening and closing unit to open the connection channel, and the volume change unit controls the opening and closing unit to close the connection channel after the ejection control unit ejects the fluid from the fluid ejection unit; and performing a second ejection operation in which the inner pressure of the fluid container is higher than a predetermined pressure, the volume change unit reduces the inner volume while controlling the opening and closing unit to open the connection channel, and the volume change unit controls the opening and closing unit to close the connection channel after the ejection control unit ejects the fluid from the fluid ejection unit.

In this manner, in the first ejection operation, since the fluid is ejected regardless of an amount of the inner pressure of the fluid container, and thereafter, the connection channel is closed, it is possible to increase the inner pressure of the fluid container to a certain amount of pressure. In addition, in the second ejection operation, it is possible to eject the fluid by using the pressure increased in the first ejection operation. The first ejection operation can be defined as an ejection operation that is performed so as to increase the inner pressure of the fluid container, and the second ejection operation can be defined as a normal ejection operation that is performed so as to eject the fluid when the inner pressure of the fluid container is increased further than the predetermined pressure. At this time, since the fluid pressure is increased by the execution of the first ejection operation, even when an inexpensive and low accuracy detectable pressure unit is adopted, it is possible to reduce an amount of time taken to eject the fluid in the second ejection operation. That is, it is possible to inexpensively reduce an amount of time taken to eject the fluid.

### Embodiment

Hereinafter, an embodiment of the invention will be described with reference to the accompanying drawings. A fluid ejection device according to the embodiment can be used in various procedures such as the cleaning or cutting of a fine object or structure, living tissue, or the like; however, an example of the embodiment given in the following description is the fluid ejection device suitable for use as an operation scalpel to incise or excise living tissue. Accordingly, a fluid used in the fluid ejecting device according to the embodiment is water, physiologic saline, a predetermined fluid medicine, or the like. The drawings referenced in the following description are schematic views in which a portion or a member is vertically and horizontally scaled differently from an actual scale for illustrative purposes.

### Entire Configuration

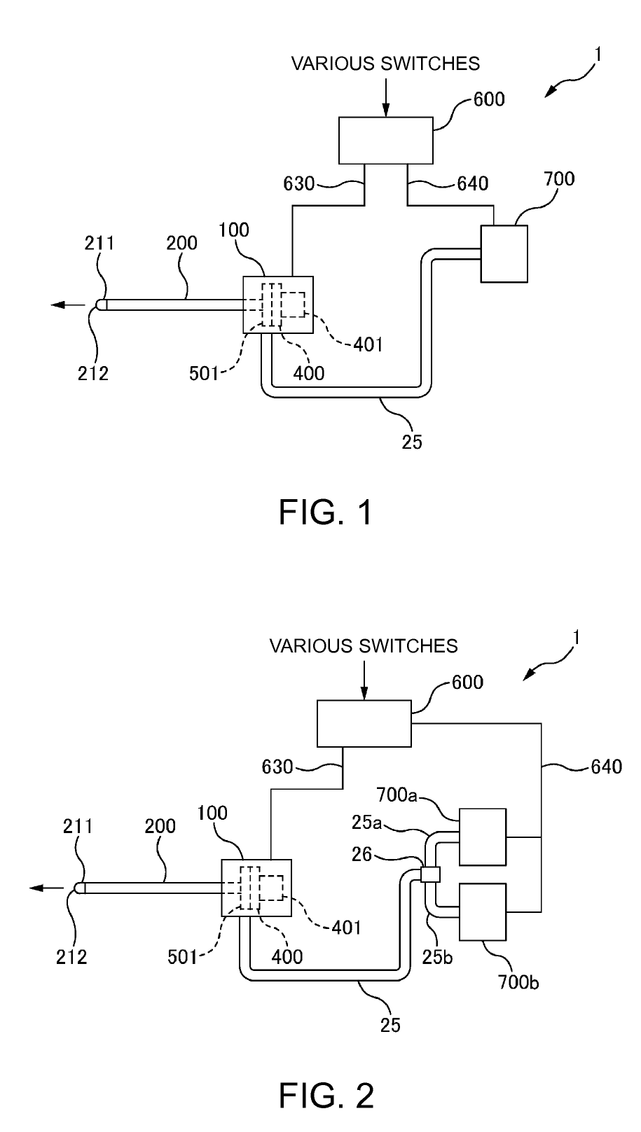

FIG. 1 is a view illustrating the configuration of a fluid ejection device 1 as an operation scalpel according to the embodiment. The fluid ejection device 1 includes a pump 700 for supplying a fluid; a pulsation generator (equivalent to a fluid ejection unit) 100 that converts the form of the fluid supplied from the pump 700 into a pulsed flow, and ejects a pulsed flow of the fluid; a drive control unit (equivalent to an ejection control unit) 600 that controls the fluid ejection device 1 in cooperation with the pump 700; and a connection tube (equivalent to a connection channel) 25 as a connection path through which the pump 700 and the pulsation generator 100 are connected to each other, and the fluid flows.

The pulsation generator 100 includes a fluid chamber 501 that accommodates the fluid supplied from the pump 700; a diaphragm 400 that changes the volume of the fluid chamber 501; and a piezoelectric element 401 that vibrates the diaphragm 400, all of which will be described later in detail.

The pulsation generator 100 includes a thin pipe-like fluid ejection tube 200 that acts as a channel of the fluid discharged from the fluid chamber 501, and a nozzle 211 that is mounted on a tip end portion of the fluid ejection tube 200 and has a reduced channel diameter.

The pulsation generator 100 converts a form of the fluid into a pulsed flow and ejects at high speed a pulsed flow of the fluid via the fluid ejection tube 200 and the nozzle 211 by driving the piezoelectric element 401 in response to drive signals output from the drive control unit 600, and changing the volume of the fluid chamber 501.

The drive control unit 600 and the pulsation generator 100 are connected to each other via a control cable 630, and drive signals for driving the piezoelectric element 401 are output from the drive control unit 600 and are transmitted to the pulsation generator 100 via the control cable 630.

The drive control unit 600 and the pump 700 are connected to each other via a communication cable 640, and the drive control unit 600 and the pump 700 transmit and receive various commands or data therebetween according to a predetermined communication protocol such as a controller area network (CAN).

The drive control unit 600 receives signals from various switches operated by a practitioner who performs an operation using the pulsation generator 100, and controls the pump 700 or the pulsation generator 100 via the control cable 630 or the communication cable 640.

The switches that input signals to the drive control unit 600 are a pulsation generator start-up switch, an ejection intensity switching switch, and the like (not illustrated).

The pulsation generator start-up switch (not illustrated) is a switch for switching a state of ejection of the fluid from the pulsation generator 100 between an ejection mode and a non-ejection mode. When a practitioner who performs an operation using the pulsation generator 100 operates the pulsation generator start-up switch (not illustrated), the drive control unit 600 controls the pulsation generator 100 to eject the fluid or stop the ejection of the fluid in cooperation with the pump 700. The pulsation generator start-up switch (not illustrated) can be a switch configured to be operated by the practitioner's feet, or a switch that is provided integrally with the pulsation generator 100 grasped by the practitioner, and configured to be operated by the practitioner's hands or fingers.

The ejection intensity switching switch (not illustrated) is a switch for changing the intensity of fluid ejection from the pulsation generator 100. When the ejection intensity switching switch (not illustrated) is operated, the drive control unit 600 controls the pulsation generator 100 and the pump 700 so as to increase and decrease the intensity of fluid ejection.

In the embodiment, a pulsed flow implies a flow of a fluid, a flow direction of which is constant, and the flow rate or flow speed of which is changed periodically or non-periodically. The pulsed flow may be an intermittent flow in which the flowing and stopping of the fluid are repeated; however, since the flow rate or flow speed of the fluid is preferably changed periodically or non-periodically, the pulsed flow is not necessarily an intermittent flow.

Similarly, the ejection of a fluid in a pulsed form implies the ejection of the fluid by which the flow rate or moving speed of an ejected fluid is changed periodically or non-periodically. An example of the pulsed ejection is an intermittent ejection by which the ejection and non-ejection of a fluid are repeated; however, since the flow rate or moving speed of an ejected fluid is preferably changed periodically or non-periodically, the pulsed ejection is not necessarily an intermittent ejection.

When the driving of the pulsation generator 100 is stopped, that is, when the volume of the fluid chamber 501 is not changed, the fluid supplied from the pump 700 as a fluid supply unit at a predetermined pressure continuously flows out of the nozzle 211 via the fluid chamber 501.

The fluid ejection device 1 according to the embodiment may be configured to include a plurality of the pumps 700.

FIG. 2 is a view illustrating the configuration of the fluid ejection device 1 configured to include two pumps 700. In this case, the fluid ejection device 1 includes a first pump 700a and a second pump 700b. A first connection tube 25a, a second connection tube 25b, the connection tube 25, and a three way stopcock 26 form a connection path which connects the pulsation generator 100 and the first pump 700a and the pulsation generator 100 and the second pump 700b, and through which the fluid flows.

The three way stopcock 26 is a valve configured to be able to communicate the first connection tube 25a and the connection tube 25, or the second connection tube 25b and the connection tube 25, and either one of the first pump 700a and the second pump 700b is selectively used.

In this configuration, for example, when the first pump 700a cannot supply the fluid for unknown reasons such as a malfunction while being selected and used, it is possible to continuously use the fluid ejection device 1 and to minimize adverse effects associated with the non-supply of the fluid from the first pump 700a by switching the three way stopcock 26 so as to communicate the second connection tube 25b and the connection tube 25, and starting the supply of the fluid from the second pump 700b.

When the fluid ejection device 1 is configured to include a plurality of the pumps 700, but the pumps 700 are not required to be distinctively described, in the following description, the pumps 700 are collectively expressed by the pump 700.

In contrast, when the plurality of pumps 700 are required to be distinctively described, suffixes such as “a” and “b” are properly added to reference sign 700 of the pump, and each of the pumps 700 is distinctively expressed by the first pump 700a or the second pump 700b. In this case, each configuration element of the first pump 700a is expressed by adding the suffix “a” to a reference sign of each configuration element, and each configuration element of the second pump 700b is expressed by adding the suffix “b” to a reference sign of each configuration element.

### Pump

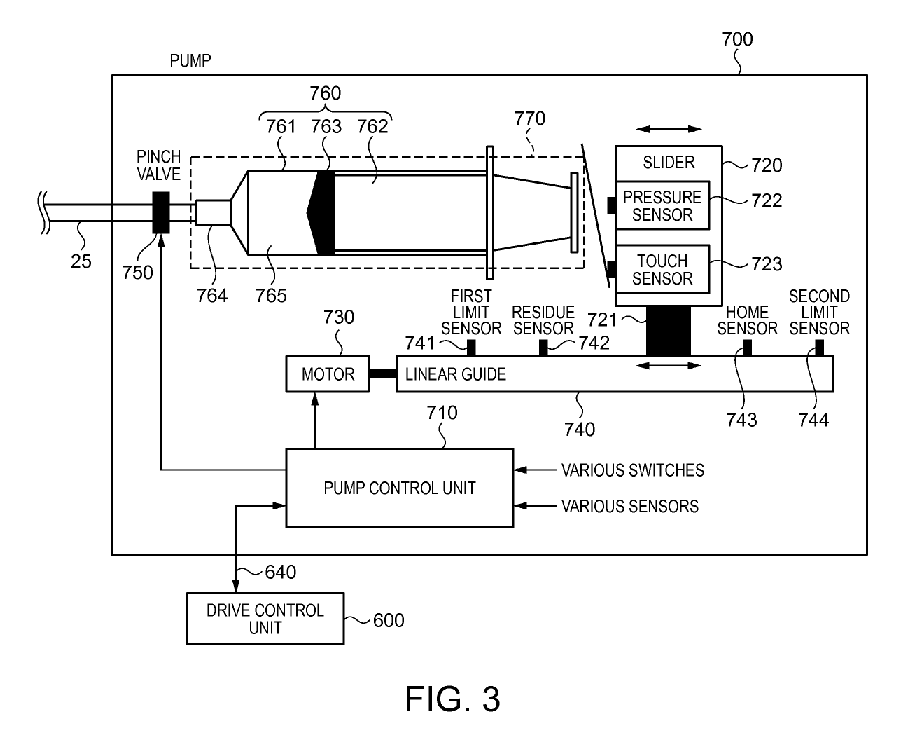

Subsequently, an outline of the configuration and operation of the pump 700 according to the embodiment will be described. FIG. 3 is a schematic view illustrating the configuration of the pump 700 according to the embodiment.

The pump 700 according to the embodiment includes a pump control unit (equivalent to a volume change unit for changing the inner volume of a fluid container) 710; a slider 720; a motor 730; a linear guide 740; and a pinch valve (equivalent to an opening and closing unit) 750. The pump 700 is configured to have a fluid container mounting unit 770 for attachably and detachably mounting a fluid container 760 that accommodates the fluid. The fluid container mounting unit 770 is formed so as to hold the fluid container 760 at a specific position when the fluid container 760 is mounted thereon.

The following switches (which will be described later in detail) (not illustrated) input signals to the pump control unit 710: a slider release switch; a slider set switch; a fluid supply switch; and a pinch valve switch.

In the embodiment, for example, the fluid container 760 is formed of a medical syringe configured to include a syringe 761 and a plunger 762.

In the fluid container 760, a protrusive cylinder-shaped opening 764 is formed in a tip end portion of the syringe 761. When the fluid container 760 is mounted on the fluid container mounting unit 770, an end portion of the connection tube 25 is inserted into the opening 764, and a fluid channel is formed from the inside of the syringe 761 to the connection tube 25.

The pinch valve 750 is a valve that is provided on a path of the connection tube 25, and opens and closes a fluid channel between the fluid container 760 and the pulsation generator 100.

The pump control unit 710 controls the opening and closing of the pinch valve 750. When the pump control unit 710 opens the pinch valve 750, the fluid container 760 and the pulsation generator 100 communicate with each other via the channel therebetween. When the pump control unit 710 closes the pinch valve 750, the channel between the fluid container 760 and the pulsation generator 100 is shut off.

In a state where the fluid container 760 is mounted on the fluid container mounting unit 770, and the pinch valve 750 is opened, when the plunger 762 of the fluid container 760 moves in a direction (hereinafter, also referred to as a push-in direction) in which the plunger 762 is pushed into the syringe 761, the volume of a space (hereinafter, also referred to as a fluid accommodation portion 765) is reduced, the space being enveloped by an end surface of a gasket 763 made of resin such as elastic rubber and mounted at the tip of the plunger 762 in the push-in direction, and an inner wall of the syringe 761, and the fluid in the fluid accommodation portion 765 is discharged via the opening 764 of the tip end portion of the syringe 761. The connection tube 25 is filled with the fluid discharged via the opening 764, and the discharged fluid is supplied to the pulsation generator 100.

In contrast, in a state where the fluid container 760 is mounted on the fluid container mounting unit 770, and the pinch valve 750 is closed, when the plunger 762 of the fluid container 760 moves in the push-in direction, it is possible to reduce the volume of the fluid accommodation portion 765, the fluid accommodating portion 765 being enveloped by the gasket 763 mounted at the tip of the plunger 762 and the inner wall of the syringe 761, and it is possible to increase the pressure of the fluid in the fluid accommodation portion 765.

The pump control unit 710 moves the slider 720 along a direction (in the push-in direction and the opposite direction of the push-in direction) in which the plunger 762 moves in a state where the fluid container 760 is mounted on the fluid container mounting unit 770, and the plunger 762 moves in accordance with the movement of the slider 720.

Specifically, the slider 720 is attached to the linear guide 740 in such a manner that a pedestal 721 of the slider 720 engages with a rail (not illustrated) formed linearly on the linear guide 740 along the slide direction of the plunger 762. The linear guide 740 moves the pedestal 721 of the slider 720 along the rail using power transmitted from the motor 730 driven by the pump control unit 710, and thereby the slider 720 moves along the slide direction of the plunger 762.

As illustrated in FIG. 3, the following sensors are provided along the rail of the linear guide 740: a first limit sensor 741; a residue sensor 742; a home sensor 743; and a second limit sensor 744.

All of the first limit sensor 741, the residue sensor 742, the home sensor 743, and the second limit sensor 744 are sensors for detecting the position of the slider 720 that moves on the rail of the linear guide 740, and signals detected by these sensors are input to the pump control unit 710.

The home sensor 743 is a sensor used to determine an initial position (hereinafter, also referred to as a home position) of the slider 720 on the linear guide 740. The home position is a position in which the slider 720 is held when the fluid container 760 is mounted or replaced.

The residue sensor 742 is a sensor for detecting the position (hereinafter, also referred to as a residual position) of the slider 720 when the residue of the fluid in the fluid container 760 is less than or equal to a predetermined value while the slider 720 moves from the home position in the push-in direction of the plunger 762. When the slider 720 reaches the residual position in which the residue sensor 742 is provided, a predetermined alarm is output to an operator (a practitioner or an assistant). The fluid container 760 currently in use is replaced with a new fluid container 760 at an appropriate time determined by the operator. Alternatively, when the second pump 700b having the same configuration as that of the pump 700 (the first pump 700a) is prepared, a switching operation is performed so as to supply the fluid from an auxiliary second pump 700b to the pulsation generator 100.

The first limit sensor 741 indicates a limit position (hereinafter, referred to as a first limit position) in a movable range in which the slider 720 can move from the home position in the push-in direction of the plunger 762. When the slider 720 reaches the first limit position in which the first limit sensor 741 is provided, the residue of the fluid in the fluid container 760 is much less than the residue indicating that the slider 720 is present at the residual position, and a predetermined alarm is output to the operator. In this case, the fluid container 760 currently in use is also replaced with a new fluid container 760, or a switching operation is also performed so as to supply the fluid from an auxiliary second pump 700b.

In contrast, the second limit sensor 744 indicates a limit position (hereinafter, also referred to as a second limit position) in a movable range in which the slider 720 can move from the home position in the opposite direction of the push-in direction of the plunger 762. When the slider 720 reaches the second limit position in which the second limit sensor 744 is provided, a predetermined alarm is output.

A touch sensor 723 and a pressure sensor 722 are mounted on the slider 720.

The touch sensor 723 is a sensor for detecting whether the slider 720 is in contact with the plunger 762 of the fluid container 760.

The pressure sensor 722 is a sensor that detects the pressure of the fluid in the fluid accommodation portion 765 formed by the inner wall of the syringe 761 and the gasket 763, and outputs signals in response to a detected pressure.

When the pinch valve 750 is closed, and the slider 720 moves in the push-in direction, and after the slider 720 comes into contact with the plunger 762, the pressure of the fluid in the fluid accommodation portion 765 increases to the extent that the slider 720 moves further in the push-in direction.

In contrast, when the pinch valve 750 is opened, and the slider 720 moves in the push-in direction, and even after the slider 720 comes into contact with the plunger 762, the fluid in the fluid accommodation portion 765 flows out of the nozzle 211 of the pulsation generator 100 via the connection tube 25, and thereby the pressure of the fluid in the fluid accommodation portion 765 increases to a certain level, but the pressure of the fluid does not increase even though the slider 720 moves further in the push-in direction.

The touch sensor 723 and the pressure sensor 722 input signals to the pump control unit 710.

A description to be given hereinafter is regarding a preparation operation configured to include a process of mounting a fluid container 760 filled with the fluid on the fluid container mounting unit 770; a process of supplying the fluid in the fluid container 760 to the pulsation generator 100; and a process of bringing the fluid ejection device 1 into a state in which the pulsation generator 100 can eject the fluid in the form of a pulsed flow.

First, the operator inputs an ON signal of the slider release switch to the pump control unit 710 by operating the slider release switch (not illustrated). Thus, the pump control unit 710 moves the slider 720 to the home position.

The operator mounts the fluid container 760 connected to the connection tube 25 in advance on the fluid container mounting unit 770. The syringe 761 of the fluid container 760 is already filled with the fluid.

When the operator sets the connection tube 25 to the pinch valve 750, and then inputs an ON signal of the pinch valve switch (not illustrated) to the pump control unit 710 by operating the pinch valve switch, the pump control unit 710 closes the pinch valve 750.

Thereafter, the fluid is ejected in a trial mode (to be described later). Accordingly, the inner pressure of the fluid container 760 is in a rough window (a range between a pressure R1 and a pressure R2) (to be described later), and the connection tube 25 or the pulsation generator 100 is filled with the fluid.

In this state, when the operator inputs an ON signal of the pulsation generator start-up switch (not illustrated) to the drive control unit 600 by operating the pulsation generator start-up switch via the feet, the pump control unit 710 opens the pinch valve 750 in response to signals transmitted from the drive control unit 600, and starts the supply of the fluid to the pulsation generator 100 by moving the slider 720 at a predetermined speed in the push-in direction at the same time or substantially the same time (for example, a time gap of approximately several milliseconds or approximately several tens of milliseconds) as when the pinch valve 750 is opened. In contrast, the drive control unit 600 generates a pulsed flow by starting the driving of the piezoelectric element 401 and changing the volume of the fluid chamber 501. Accordingly, a pulsed flow of the fluid is ejected at a high speed via the nozzle 211 at the tip of the pulsation generator 100 (the ejection of the fluid in a normal ejection mode).

Thereafter, when the operator inputs an OFF signal of the pulsation generator start-up switch (not illustrated) to the drive control unit 600 by operating the pulsation generator start-up switch via the feet, the drive control unit 600 stops the driving of the piezoelectric element 401. The pump control unit 710 stops the movement of the slider 720 in response to signals transmitted from the drive control unit 600, and closes the pinch valve 750. As such, the pulsation generator 100 stops the ejection of the fluid.

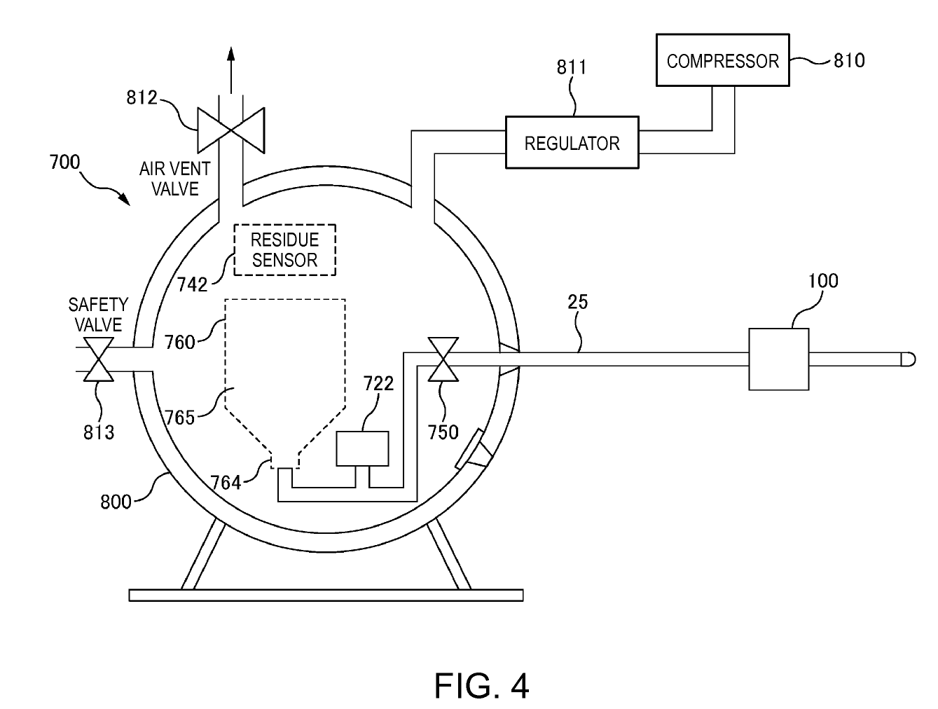

The pump 700 according to the embodiment is configured such that the slider 720 presses the fluid container 760 that is formed of a medical syringe configured to include the syringe 761 and the plunger 762; however, the pump 700 may be configured as illustrated in FIG. 4.

FIG. 4 is a view illustrating the pump 700 with a different configuration. The pump 700 illustrated in FIG. 4 has the following configuration: the fluid container 760 (an infusion solution bag that accommodates a fluid) is mounted in a pressurized chamber 800, and after air supplied from a compressor 810 is regulated by a regulator 811, the air is pressure-fed into the pressurized chamber 800, and thereby the fluid container 760 is pressed.

When the pinch valve 750 is opened in a state where the fluid container 760 is pressed by the pressurization of air in the pressurized chamber 800, the fluid accommodated in the fluid accommodation portion 765 of the fluid container 760 flows out of the opening 764, and is supplied to the pulsation generator 100 via the connection tube 25.

The air in the pressurized chamber 800 is released to the atmosphere by the opening of an air vent valve 812. In a case where the pressure of the air in the pressurized chamber 800 exceeds a predetermined pressure, even when the air vent valve 812 is not opened, a safety valve 813 is opened, and thereby the air in the pressurized chamber 800 is released to the atmosphere.

The pump control unit 710 controls the compressor 810; the regulator 811; the air vent valve 812; and the pinch valve 750, the control scheme of which is not illustrated in FIG. 4. The following sensors input detected output signals to the pump control unit 710: the pressure sensor 722 that detects the pressure of the fluid in the fluid container 760, and the residue sensor 742 that detects the residue of the fluid in the fluid container 760.

When the pump 700 with this configuration is adopted, it is possible to increase the amount of the fluid which can be supplied to the pulsation generator 100 per unit time. Since the pulsation generator 100 can supply the fluid at a high pressure, and an infusion solution bag that accommodates the fluid is used as the fluid container 760 as it is, it is possible to prevent the fluid from being contaminated. The pulsation generator 100 can continuously supply the fluid without generating pulsation.

In addition, in the embodiment, the drive control unit 600 is provided separately from the pump 700 and the pulsation generator 100; however, the drive control unit 600 may be provided integrally with the pump 700.

When the practitioner performs an operation using the fluid ejection device 1, the practitioner grasps the pulsation generator 100. Accordingly, the connection tube 25 up to the pulsation generator 100 is preferably as flexible as possible. For this reason, a flexible thin tube is used as the connection tube 25, and a fluid discharge pressure of the pump 700 is preferably set to a low pressure in a pressure range in which the fluid can be supplied to the pulsation generator 100. For this reason, the discharge pressure of the pump 700 is set to approximately 0.3 atm (0.03 MPa) or less.

In particular, in a case where a malfunction of an apparatus may lead to a serious accident, for example, for brain surgery, it is necessary to prevent the cutting of the connection tube 25 from causing the ejection of the fluid at a high pressure, and also, for this reason, the discharge pressure of the pump 700 is required to be set to a low pressure.

### Pulsation Generator

Subsequently, the structure of the pulsation generator 100 according to the embodiment will be described.

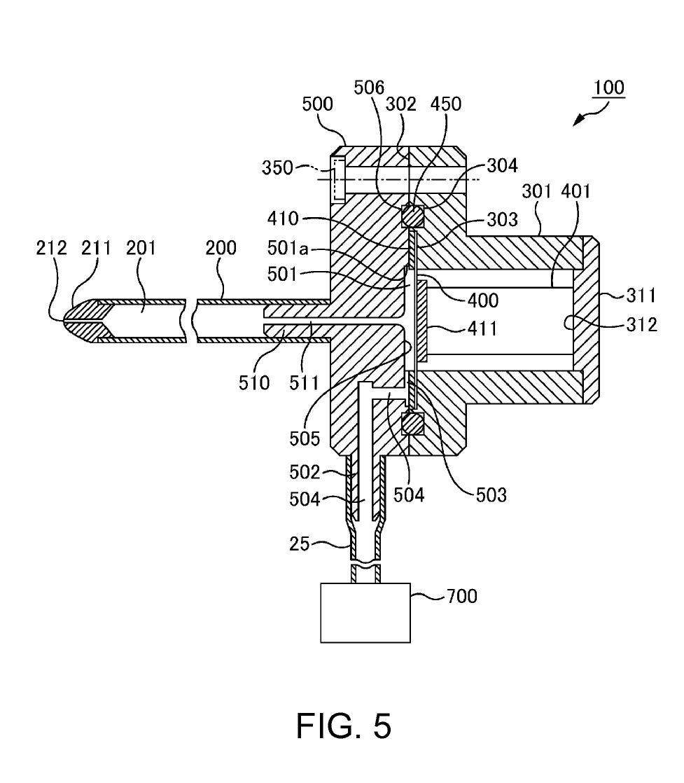

FIG. 5 is a cross-sectional view illustrating the structure of the pulsation generator 100 according to the embodiment. In FIG. 5, the pulsation generator 100 includes a pulse generation unit that generates the pulsation of the fluid, and is connected to the fluid ejection tube 200 having a connection channel 201 as a channel through which the fluid is discharged.

In the pulsation generator 100, an upper case 500 and a lower case 301 are screwed together with four fixation screws 350 (not illustrated) while the respective facing surfaces thereof are bonded to each other. The lower case 301 is a cylindrical member having a flange, and one end portion of the lower case 301 is sealed with a bottom plate 311. The piezoelectric element 401 is provided in an inner space of the lower case 301.

The piezoelectric element 401 is a stack-type piezoelectric element, and acts as an actuator. One end portion of the piezoelectric element 401 is firmly fixed to the diaphragm 400 via an upper plate 411, and the other end portion is firmly fixed to an upper surface 312 of the bottom plate 311.

The diaphragm 400 is made of a circular disc-like thin metal plate, and a circumferential edge portion of the diaphragm 400 is firmly fixed to a bottom surface of a concave portion 303 in the lower case 301 while being in close contact with the bottom surface of the concave portion 303. When drive signals are input to the piezoelectric element 401 that acts as a volume change unit, the piezoelectric element 401 changes the volume of the fluid chamber 501 via the diaphragm 400 through the extension and contraction thereof.

A reinforcement plate 410 is provided in such a manner as to be stacked on an upper surface of the diaphragm 400, and is made of a circular disc-like thin metal plate having an opening at the center thereof.

The upper case 500 has a concave portion formed in a center portion of the surface facing the lower case 301, and the fluid chamber 501 is a rotator-shaped space formed by this concave portion and the diaphragm 400 and filled with the fluid. That is, the fluid chamber 501 is a space enveloped by a sealing surface 505 and an inner circumferential side wall 501a of the concave portion of the upper case 500, and the diaphragm 400. An outlet channel 511 is drilled in an approximately center portion of the fluid chamber 501.

The outlet channel 511 passes through the outlet channel tube 510 from the fluid chamber 501 to an end portion of an outlet channel tube 510 provided in such a manner as to protrude from one end surface of the upper case 500. A connection portion between the outlet channel 511 and the sealing surface 505 of the fluid chamber 501 is smoothly rounded so as to reduce fluid resistance.

In the embodiment (refer to FIG. 5), the fluid chamber 501 has a substantially cylindrical shape having sealed opposite ends; however, the fluid chamber 501 may have a conical shape, a trapezoidal shape, a hemispherical shape, or the like in a side view, and the shape of the fluid chamber 501 is not limited to a cylindrical shape. For example, when the connection portion between the outlet channel 511 and the sealing surface 505 has a funnel shape, air bubbles in the fluid chamber 501 (to be described later) are easily discharged.

The fluid ejection tube 200 is connected to the outlet channel tube 510. The connection channel 201 is drilled in the fluid ejection tube 200, and the diameter of the connection channel 201 is larger than that of the outlet channel 511. In addition, the tube thickness of the fluid ejection tube 200 is formed so as to have a range of rigidity in which the fluid ejection tube 200 does not absorb pressure pulsation of the fluid.

The nozzle 211 is inserted into the tip end portion of the fluid ejection tube 200. A fluid ejection opening 212 is drilled in the nozzle 211. The diameter of the fluid ejection opening 212 is smaller than that of the connection channel 201.

An inlet channel tube 502 is provided in such a manner as to protrude from a side surface of the upper case 500, and is inserted into the connection tube 25 through which the fluid is supplied from the pump 700. A connection channel 504 for the inlet channel is drilled in the inlet channel tube 502. The connection channel 504 communicates with an inlet channel 503. The inlet channel 503 is formed in a groove shape in a circumferential edge portion of the sealing surface 505 of the fluid chamber 501, and communicates with the fluid chamber 501.

A packing box 304 and a packing box 506 are respectively formed in the bonded surfaces of the lower case 301 and the upper case 500 at positions separated from an outer circumferential direction of the diaphragm 400, and a ring-shaped packing 450 is mounted in a space formed by the packing boxes 304 and 506.

Here, when the upper case 500 and the lower case 301 are assembled together, the circumferential edge portion of the diaphragm 400 is in close contact with a circumferential edge portion of the reinforcement plate 410 due to the circumferential edge portion of the sealing surface 505 of the upper case 500 and the bottom surface of the concave portion 303 of the lower case 301. At this time, the packing 450 is pressed by the upper case 500 and the lowercase 301, and thereby the fluid is prevented from leaking from the fluid chamber 501.

Since the inner pressure of the fluid chamber 501 becomes a high pressure of 30 atm (3 MPa) or greater during the discharge of the fluid, the fluid may slightly leak from the respective connections between the diaphragm 400, the reinforcement plate 410, the upper case 500, and the lower case 301; however, the leakage of the fluid is prevented due to the packing 450.

As illustrated in FIG. 5, in the case where the packing 450 is provided, since the packing 450 is compressed due to the pressure of the fluid leaking from the fluid chamber 501 at a high pressure, and is strongly pressed against the respective walls of the packing boxes 304 and 506, it is possible to more reliably prevent the leakage of the fluid. For this reason, it is possible to maintain a considerable increase in the inner pressure of the fluid chamber 501 during the driving of the pulsation generator 100.

Subsequently, the inlet channel 503 formed in the upper case 500 will be described with reference to the drawings in more detail.

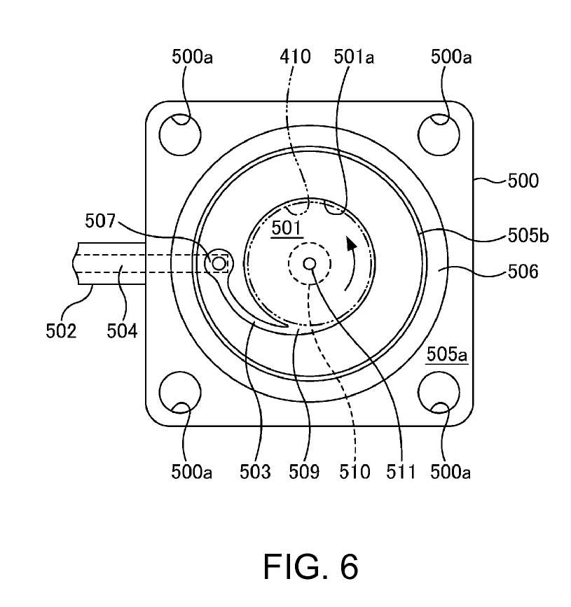

FIG. 6 is a plan view illustrating the shape of the inlet channel 503. FIG. 6 illustrates the shape of the upper case 500 when the surface of the upper case 500 bonded to the lower case 301 is seen.

In FIG. 6, the inlet channel 503 is formed in a groove shape in the circumferential edge portion of the sealing surface 505 of the upper case 500.

One end portion of the inlet channel 503 communicates with the fluid chamber 501, and the other end portion communicates with the connection channel 504. A fluid sump 507 is formed in a connection portion between the inlet channel 503 and the connection channel 504. A connection portion between the fluid sump 507 and the inlet channel 503 is smoothly rounded, and thereby fluid resistance is reduced.

The inlet channel 503 communicates with the fluid chamber 501 in a substantially tangential direction with respect to an inner circumferential side wall 501a of the fluid chamber 501. The fluid supplied from the pump 700 (refer to FIG. 1) at a predetermined pressure flows along the inner circumferential side wall 501a (in a direction illustrated by the arrow in FIG. 6), and generates a swirl flow in the fluid chamber 501. The swirl flow is pushed against the inner circumferential side wall 501a due to a centrifugal force associated with the swirling of the fluid, and air bubbles in the fluid chamber 501 are concentrated in a center portion of the swirl flow.

The air bubbles concentrated in the center portion are discharged via the outlet channel 511. For this reason, the outlet channel 511 is preferably provided in the vicinity of the center of the swirl flow, that is, in an axial center portion of a rotor shape.

As illustrated in FIG. 6, the inlet channel 503 is curved. The inlet channel 503 may communicate with the fluid chamber 501 while not being curved but being linearly formed; however, when the inlet channel 503 is curved, a channel length is increased, and a desired inertance (to be described later) is obtained in a small space.

As illustrated in FIG. 6, the reinforcement plate 410 is provided between the diaphragm 400 and the circumferential edge portion of the sealing surface 505, in which the inlet channel 503 is formed. The reinforcement plate 410 is provided so as to improve the durability of the diaphragm 400. Since a cut-out connection opening 509 is formed in a connection portion between the inlet channel 503 and the fluid chamber 501, when the diaphragm 400 is driven at a high frequency, stress may be concentrated in the vicinity of the connection opening 509, and thereby a fatigue failure may occur in the vicinity of the connection opening 509. It is possible to prevent stress from being concentrated on the diaphragm. 400 by providing the reinforcement plate 410 with an opening not having a cut-out portion and being continuously formed.

Four screw holes 500a are respectively provided in outer circumferential corner portions of the upper case 500, and the upper case 500 and the lower case 301 are bonded to each other via screwing at the positions of the screw holes.

It is possible to firmly fix the reinforcement plate 410 and the diaphragm 400 in an integrally stacked state by bonding together the reinforcement plate 410 and the diaphragm 400, which is not illustrated. An adhesive method using an adhesive, a solid-state diffusion bonding method, a welding method, or the like may be used so as to firmly fix together the reinforcement plate 410 and the diaphragm 400; however, the respective bonded surfaces of the reinforcement plate 410 and the diaphragm 400 are preferably in close contact with each other.

### Operation of Pulsation Generator

Subsequently, an operation of the pulsation generator 100 according to the embodiment will be described with reference to FIGS. 1 to 6. The pulsation generator 100 according to the embodiment discharges the fluid due to a difference between an inertance L1 (may be referred to as a combined inertance L1) of the inlet channel 503 and the peripherals and an inertance L2 (may be referred to as a combined inertance L2) of the outlet channel 511 and the peripherals.

### Inertance

First, the inertance will be described.

An inertance L is expressed by L=ρ×h/S, and here, ρ is the density of a fluid, S is the cross-sectional area of a channel, and h is a channel length. When ΔP is a differential pressure of the channel, and Q is a flow rate of the fluid flowing through the channel, it is possible to deduce a relationship ΔP=L×dQ/dt by modifying an equation of motion in the channel using the inertance L.

That is, the inertance L indicates a degree of influence on a change in flow rate with time, and a change in flow rate with time decreases to the extent that the inertance L is large, and a change in flow rate with time increases to the extent that the inertance L is small.

Similar to a parallel connection or a series connection of inductances in an electric circuit, it is possible to calculate a combined inertance with respect to a parallel connection of a plurality of channels or a series connection of a plurality of channels having different shapes by combining an inertance of each of the channels.

Since the diameter of the connection channel 504 is set to be larger much than that of the inlet channel 503, the inertance L1 of the inlet channel 503 and the peripherals can be calculated from a boundary of the inlet channel 503. At this time, since the connection tube 25 that connects the pump 700 and the inlet channel 503 is flexible, the connection tube 25 may not be taken into consideration in calculating the inertance L1.

Since the diameter of the connection channel 201 is larger much than that of the outlet channel 511, and the tube (tube wall) thickness of the fluid ejection tube 200 is thin, the connection tube 25 and the fluid ejection device 1 have a negligible influence on the inertance L2 of the outlet channel 511 and the peripherals. Accordingly, the inertance L2 of the outlet channel 511 and the peripherals may be replaced with an inertance of the outlet channel 511.

The rigidity of the tube wall thickness of the fluid ejection tube 200 is sufficient to propagate the pressure of the fluid.

In the embodiment, a channel length and a cross-sectional area of the inlet channel 503 and a channel length and a cross-sectional area of the outlet channel 511 are set in such a manner that the inertance L1 of the inlet channel 503 and the peripherals are greater than the inertance L2 of the outlet channel 511 and the peripherals.

### Ejection of Fluid

Subsequently, an operation of the pulsation generator 100 will be described.

The pump 700 supplies the fluid to the inlet channel 503 at a predetermined pressure. As a result, when the piezoelectric element 401 is not operated, the fluid flows into the fluid chamber 501 due to a difference between a discharge force of the pump 700 and a fluid resistance value for the entirety of the inlet channel 503 and the peripherals.

Here, in a case where the inertance L1 of the inlet channel 503 and the peripherals and the inertance L2 of the outlet channel 511 and the peripherals are considerably large, when a drive signal is input to the piezoelectric element 401, and the piezoelectric element 401 extends rapidly, the inner pressure of the fluid chamber 501 increases rapidly, and reaches several tens of atmosphere.

Since the inner pressure of the fluid chamber 501 is larger much than the pressure applied to the inlet channel 503 by the pump 700, the flow of the fluid from the inlet channel 503 to the fluid chamber 501 decreases due to the pressure, and the flow of the fluid out of the outlet channel 511 increases.

Since the inertance L1 of the inlet channel 503 is larger than the inertance L2 of the outlet channel 511, an increase in a flow rate of the fluid discharged from the outlet channel 511 is larger than a decrease in a flow rate of the fluid flowing from the inlet channel 503 into the fluid chamber 501. Accordingly, the fluid is discharged in the form of a pulsed flow to the connection channel 201, that is, a pulsed flow occurs. Discharge pressure pulsation propagates in the fluid ejection tube 200, and the fluid is ejected via the fluid ejection opening 212 of the nozzle 211 at the tip end.

Here, since the diameter of the fluid ejection opening 212 of the nozzle 211 is smaller than that of the outlet channel 511, a pulsed flow of the fluid is ejected as droplets at a higher pressure and speed.

In contrast, immediately after a pressure increase, the inner pressure of the fluid chamber 501 becomes negative due to interaction between a decrease in the amount of inflow of the fluid from the inlet channel 503 and an increase in the amount of outflow of the fluid from the outlet channel 511. As a result, after a predetermined amount of time has elapsed, due to both of the pressure of the pump 700 and the negative inner pressure of the fluid chamber 501, the fluid flows from the inlet channel 503 into the fluid chamber 501 again at the same speed as that before the operation of the piezoelectric element 401.

When the piezoelectric element 401 extends after the flow of the fluid from the inlet channel 503 is restored, it is possible to continuously eject the fluid in the form of a pulsed flow via the nozzle 211.

### Discharge of Air Bubbles

Subsequently, an operation of discharging air bubbles from the fluid chamber 501 will be described.

As described above, the inlet channel 503 communicates with the fluid chamber 501 via a path that approaches the fluid chamber 501 while swirling around the fluid chamber 501. The outlet channel 511 is provided in the vicinity of a rotational axis of a substantially rotor-shaped fluid chamber 501.

For this reason, the fluid flowing from the inlet channel 503 into the fluid chamber 501 swirls along the inner circumferential side wall 501a of the fluid chamber 501. The fluid is pushed against the inner circumferential side wall 501a of the fluid chamber 501 due to a centrifugal force, and air bubbles contained in the fluid are concentrated in the center portion of the fluid chamber 501, and are discharged via the outlet channel 511.

Accordingly, even when a small amount of the volume of the fluid chamber 501 is changed in association with the operation of the piezoelectric element 401, it is possible to obtain a sufficient pressure increase while a pressure pulsation is not adversely affected by the air bubbles.

In the embodiment, since the pump 700 supplies the fluid to the inlet channel 503 at a predetermined pressure, even when the driving of the pulsation generator 100 is stopped, the fluid is supplied to the inlet channel 503 and the fluid chamber 501. Accordingly, it is possible to start an initial operation without an aid of a prime operation.

Since the fluid is ejected via the fluid ejection opening 212 having a diameter smaller than that of the outlet channel 511, an inner fluid pressure increases higher than that of the outlet channel 511, and thereby it is possible to eject the fluid at a high speed.

Since the rigidity of the fluid ejection tube 200 is sufficient to transmit a pulsation of the fluid from the fluid chamber 501 to the fluid ejection opening 212, it is possible to eject the fluid in the form of a desired pulsed flow without disturbing pressure propagation of the fluid from the pulsation generator 100.

Since the inertance of the inlet channel 503 is set to be larger than that of the outlet channel 511, an increase in the amount of outflow of the fluid from the outlet channel 511 is larger than a decrease in the amount of inflow of the fluid from the inlet channel 503 into the fluid chamber 501, and it is possible to discharge the fluid into the fluid ejection tube 200 in the form of a pulsed flow. Accordingly, a check valve is not required to be provided in the inlet channel 503, it is possible to simplify the structure of the pulsation generator 100, it is easy to clean the inside of the pulsation generator 100, and it is possible to remove a potential durability problem associated with the use of the check valve.

Since the respective inertances of both of the inlet channel 503 and the outlet channel 511 are set to be considerably large, it is possible to rapidly increase the inner pressure of the fluid chamber 501 by rapidly reducing the volume of the fluid chamber 501.

Since the piezoelectric element 401 as a volume change unit and the diaphragm 400 are configured so as to generate a pulsation, it is possible to simplify the structure of the pulsation generator 100 and to reduce the size of the pulsation generator 100 in association therewith. It is possible to set the maximum frequency of a change in the volume of the fluid chamber 501 to a high frequency of 1 KHz or greater, and the pulsation generator 100 is optimized to eject a pulsed flow of the fluid at a high speed.

In the pulsation generator 100, since the inlet channel 503 generates a swirl flow of the fluid in the fluid chamber 501, the fluid in the fluid chamber 501 is pushed in an outer circumferential direction of the fluid chamber 501 due to a centrifugal force, air bubbles contained in the fluid are concentrated in the center portion of the swirl flow, that is, in the vicinity of the axis of the substantially rotor shape, and thereby it is possible to discharge the air bubbles via the outlet channel 511 provided in the vicinity of the axis of the substantially rotor shape. For this reason, it is possible to prevent a decrease in pressure amplitude associated with the stagnation of air bubbles in the fluid chamber 501, and it is possible to continuously and stably drive the pulsation generator 100.

Since the inlet channel 503 is formed in such a manner as to communicate with the fluid chamber 501 via the path that approaches the fluid chamber 501 while swirling around the fluid chamber 501, it is possible to generate a swirl flow without adopting a structure dedicated for swirling the fluid in the fluid chamber 501.

Since the groove-shaped inlet channel 503 is formed in the outer circumferential edge portion of the sealing surface 505 of the fluid chamber 501, it is possible to form the inlet channel 503 (a swirl flow generation unit) without increasing the number of components.

Since the reinforcement plate 410 is provided on the upper surface of the diaphragm 400, the diaphragm 400 is driven with respect to an outer circumference (a fulcrum) of the opening of the reinforcement plate 410, and thereby the concentration of stress is unlikely to occur, and it is possible to improve the durability of the diaphragm 400.

When corners of the surface of the reinforcement plate 410 bonded to the diaphragm 400 are rounded, it is possible to further reduce the concentration of stress on the diaphragm 400.

When the reinforcement plate 410 and the diaphragm 400 are firmly and integrally fixed together while being stacked on each other, it is possible to improve the assemblability of the pulsation generator 100, and it is possible to reinforce the outer circumferential edge portion of the diaphragm 400.

Since the fluid sump 507 for the stagnation of the fluid is provided in the connection portion between the connection channel 504 on an inlet side for supplying the fluid from the pump 700 and the inlet channel 503, it is possible to prevent the inertance of the connection channel 504 from affecting the inlet channel 503.

In the respective bonded surfaces of the lower case 301 and the upper case 500, the ring-shaped packing 450 is provided at the position separated from the outer circumferential direction of the diaphragm 400, and thereby it is possible to prevent the leakage of the fluid from the fluid chamber 501, and to prevent a decrease in the inner pressure of the fluid chamber 501.

When it is possible to adopt a highly reliable pressure sensor or a highly accurate detectable pressure sensor as the pressure sensor 722 of the fluid container 760, it is possible to increase the inner pressure of the fluid container 760 to a necessary amount of pressure based on a pressure detected by the pressure sensor.

However, for example, a relatively non-highly reliable pressure sensor or a non-highly accurate detectable pressure sensor may be adopted as the pressure sensor 722 so as to reduce manufacturing costs. In this case, the reliability of the detected pressure is not high. Accordingly, when the inner pressure of the fluid container 760 is increased to the necessary amount of pressure based on the detected pressure, the pressure of the fluid container 760 may become an unexpectedly high pressure.

In contrast, even when such an inexpensive pressure sensor is adopted, it is desirable to eject the fluid at a proper intensity immediately after the inner pressure of the fluid container 760 is increased to a proper amount of pressure. That is, it is desirable to inexpensively reduce an amount of time taken to eject the fluid.

In a fluid ejection control operation which will be described below, when such an inexpensive pressure sensor 722 is used, it is possible to reduce an amount of time taken to eject the fluid.

Subsequently, the fluid ejection control operation will be described.

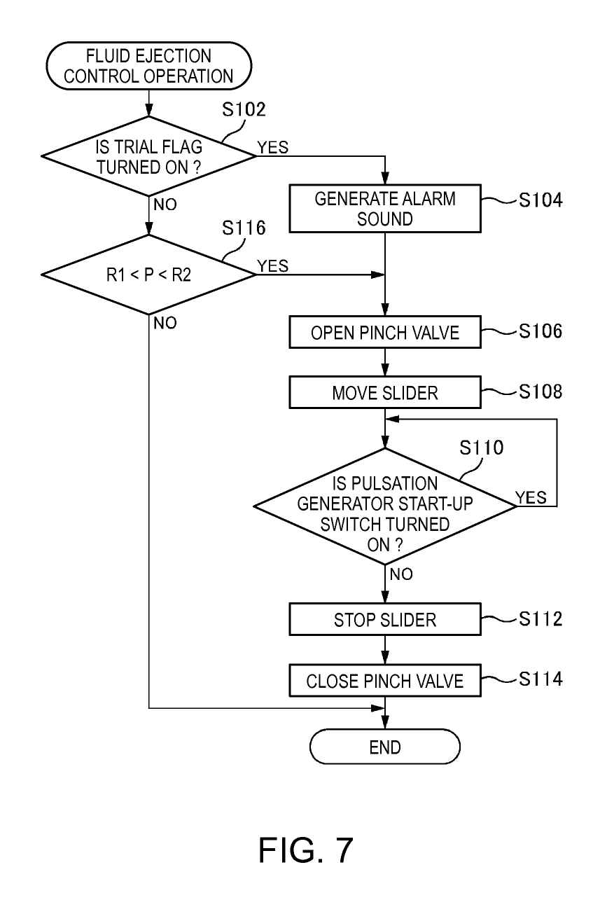

FIG. 7 is a flowchart of the fluid ejection control operation. In the following process, necessary communication is properly performed between the drive control unit 600 and the pump control unit 710. Necessary information is exchanged between the drive control unit 600 and the pump control unit 710, and the drive control unit 600 and the pump control unit 710 control each part based on this information.

In the embodiment, it is determined whether an ejection mode is the trial mode (equivalent to a first ejection mode) or the normal ejection mode (equivalent to a second ejection mode) based on a state of a trial flag (to be described later). When the trial flag is turned on, the ejection mode becomes the trial mode.

The trial flag is automatically turned on or off by being interrupted every predetermine time period, or based on a pressure detected by the pressure sensor 722. That is, separately from the flowchart of the fluid ejection control operation that will be described subsequently, the trial flag is turned on or off by monitoring the pressure detected by the pressure sensor 722. Here, when the pressure detected by the pressure sensor 722 is higher than the pressure R1 and less than the pressure R2, the trial flag is turned off, and in other cases, the trial flag is turned on. As described above, since the inexpensive pressure sensor 722 is adopted, the trial flag may be turned on or off in response to a non-highly reliable detected pressure.

When the fluid ejection control operation starts, it is determined whether the trial flag is turned on (S102). When the trial flag is turned on, a predetermined alarm sound is generated (S104). Instead of generating the predetermined alarm sound, a display unit (not illustrated) may display a message that the trial flag is turned on.

Subsequently, the drive control unit 600 instructs the pump control unit 710 to open the pinch valve 750 (S106). The drive control unit 600 instructs the pump control unit 710 to move the slider 720 at a predetermined speed in a direction in which the fluid is pushed (S108). Accordingly, the drive control unit 600 drives the piezoelectric element 401. As a result, the fluid is ejected via the nozzle 211.

Subsequently, the drive control unit 600 determines whether the pulsation generator start-up switch is continuously turned on (S110). When the pulsation generator start-up switch is continuously turned on, the process returns to step S110 again. This loop is repeated, and thereby it is possible to eject the fluid from the pulsation generator 100.

When the pulsation generator start-up switch is turned off in step S110, the drive control unit 600 stops the driving of the piezoelectric element 401. At substantially the same time, the drive control unit 600 instructs the pump control unit 710 to stop the movement of the slider 720 (S112) and to close the pinch valve 750 (S114).

In contrast, when the trial flag is turned off in step S102, it is determined whether an inner pressure P of the fluid container 760 is higher than the lower limit pressure R1, and is lower than the upper limit pressure R2 (S116). When the pressure P is higher than the lower limit pressure R1, and is lower than the upper limit pressure R2, the process moves to step S106, and the fluid is ejected.

When the pressure P is not higher than the lower limit pressure R1, or is not lower than the upper limit pressure R2 in step S116, the process ends.

In this manner, in the trial mode, since the fluid is ejected regardless of an amount of the inner pressure of the fluid container, and thereafter the connection channel is closed, it is possible to increase the inner pressure of the fluid container to a certain amount of pressure. In the normal ejection mode, it is possible to eject the fluid by using the pressure increased in the trial mode. The trial mode can be defined as an ejection mode that is enabled so as to increase the inner pressure of the fluid container, and the normal ejection mode can be defined as a normal ejection mode that is enabled so as to eject the fluid when the inner pressure of the fluid container is increased further than a predetermined pressure. At this time, since the fluid pressure is increased by the execution of the trial mode, even when the inexpensive and low accuracy detectable pressure sensor 722 is adopted, it is possible to reduce an amount of time taken to eject the fluid in the normal ejection mode. That is, it is possible to inexpensively reduce an amount of time taken to eject the fluid.

For the following reason, it is determined whether the inner pressure exceeds the upper limit pressure R2 based on a pressure detected by the inexpensive pressure sensor 722. That is, there is a problem in that the fluid may be ejected at an unexpected intensity when the inner pressure of the fluid container 760 is increased further than necessary. The ejection of the fluid at a low intensity has a small influence on the excision of living tissue or the like, and in contrast, the ejection of the fluid at a high intensity may have a great influence. For this reason, a pressure is detected by the relatively inexpensive pressure sensor 722, and it is determined whether the pressure exceeds the upper limit pressure R2 based on the detected pressure. Taking this factor into consideration, it is desirable to set the upper limit pressure R2 to a slightly low pressure.

When the pressure P is the upper limit pressure R2 or higher, a fine pressure decrease adjustment control operation may be performed. The pump control unit 710 according to the embodiment can control the motor 730 to continuously move the slider 720 at a predetermined speed, and can control the motor 730 to move the slider 720 by a very small distance. The motor 730 is controlled to rotate by a minimum unit so as to move the slider 720 by the very small distance. In the fine pressure decrease adjustment control operation, the pump control unit 710 moves the slider 720 toward the second limit sensor 744 by the very small distance. As a result, due to the inner pressure of the fluid container 760, the plunger 762 moves by the very small distance in an increase direction of the inner volume of the fluid accommodation portion 765. Accordingly, the inner pressure of the fluid container 760 decreases by a very small amount of pressure. When the fine pressure decrease adjustment control operation is completed once, it is determined whether the pressure P is the upper limit pressure P2 or higher, again. When the pressure P is the upper limit pressure R2 or higher, the fine pressure decrease adjustment control operation is performed again.

When the fluid ejection control operation is performed, the inner pressure of the fluid container 760 is changed in the following manner.

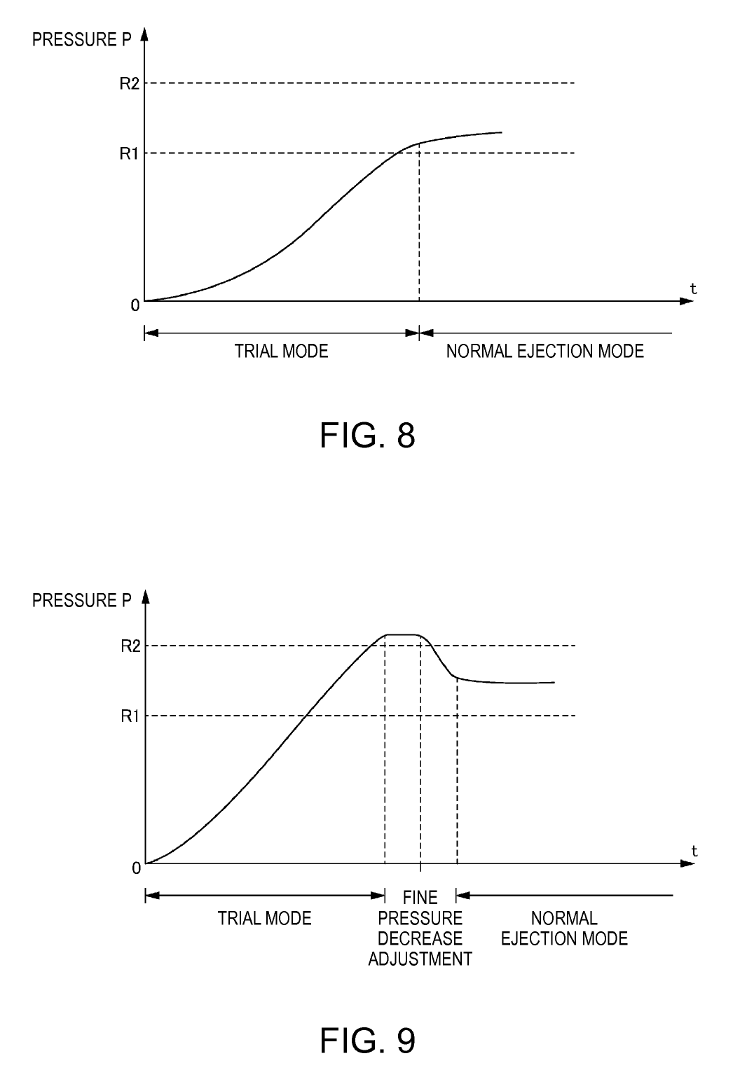

FIG. 8 is a first graph illustrating a change in pressure in the trial mode and the normal ejection mode. FIG. 8 illustrates the inner pressure P of the fluid container 760, the lower limit pressure R1, and the upper limit pressure R2. The fluid ejection device 1 according to the embodiment ejects the fluid at a desired intensity when the inner pressure P of the fluid container 760 is between the lower limit pressure R1 and the upper limit pressure R2.

The fluid ejection device 1 according to the embodiment is mechanically designed in such a manner that the inner pressure P of the fluid container 760 is between the lower limit pressure R1 and the upper limit pressure R2 when the fluid is ejected in the trial mode. For example, this design may be done in such a manner that the inner pressure P of the fluid container 760 is balanced between the lower limit pressure R1 and the upper limit pressure R2 based on a relationship between the channel resistance of the nozzle 211 and the movement speed of the slider 720. Accordingly, even when the pressure sensor 722 does not have high detection accuracy, it is possible to increase the inner pressure of the fluid container from the pressure R1 to the pressure R2.

As soon as the fluid container 760 is set to the pump 700, the plunger 762 is not pushed yet. Accordingly, the connection tube 25 is not filled with the fluid, and the inner pressure of the fluid container 760 is almost not increased. That is, as soon as the fluid container 760 is set to the pump 700, the inner pressure of the fluid container 760 is zero.

Thereafter, when the pulsation generator start-up switch is turned on, the pinch valve 750 is opened, and the slider 720 moves in the direction in which the fluid is pushed. Since the piezoelectric element 401 is also driven, the pulsation generator 100 ejects the fluid.

As described above, when the slider 720 moves in a state where the inner pressure P of the fluid container 760 is lower than the lower limit pressure R1, the inner pressure P of the fluid container 760 is increased, and finally, the pressure P is between the lower limit pressure R1 and the upper limit pressure R2. Thereafter, once the pulsation generator start-up switch is turned off, and then is turned on, the ejection mode becomes the normal ejection mode, and it is possible to eject the fluid in a state where the inner pressure of the fluid container 760 is increased to an amount of pressure required to normally eject the fluid.

That is, since the fluid is ejected in the trial mode, the inner pressure of the fluid container 760 is increased to an amount of pressure (a pressure between the lower limit pressure R1 and the upper limit pressure R2) at which the fluid can be ejected. Accordingly, when the pulsation generator start-up switch is turned on, it is possible to immediately eject the fluid. In addition, it is possible to reduce an amount of time taken to eject the fluid.

FIG. 9 is a second graph illustrating a change in pressure in the trial mode and the normal ejection mode. Similar to FIG. 8, the inner pressure of the fluid container 760 is increased in the trial mode in FIG. 9. However, in the trial mode, the inner pressure of the fluid container 760 may not exceed the upper limit pressure R2 for unknown reasons.

When the pressure P of the fluid container 760 exceeds the upper limit pressure R2, and the fluid is ejected from the pulsation generator 100, as described above, the fluid may be ejected via the nozzle 211 at an unexpected intensity due to a high pressure of the fluid supplied from the fluid container 760, which is a problem. For this reason, when the inner pressure of the fluid container 760 exceeds the upper limit pressure R2, it is possible to perform the fine pressure decrease adjustment control operation.

Accordingly, the inner pressure P of the fluid container 760 becomes less than the upper limit pressure R2.

In the fine pressure decrease adjustment control operation, since the pressure is slightly decreased, the pressure P is maintained higher than the lower limit pressure R1.

### Another Embodiment

In the example of the embodiment, the fluid ejection device 1 is applied to an operation scalpel used to incise or excise living tissue; however, the invention is not limited to the embodiment, and can be applied to other medical tools for excision, cleaning, or the like. Specifically, the fluid ejection device 1 can be used to clean a fine object or structure.

In the embodiment, the fluid is ejected by using the piezoelectric element; however, a laser bubble method may be adopted by which a fluid in a pressure chamber is powerfully ejected by generating bubbles in the fluid in the pressure chamber with a laser beam. A heater bubble method may be adopted by which a fluid in a pressure chamber is powerfully ejected by generating bubbles in the fluid in the pressure chamber with a heater.

In the embodiment, the fluid is ejected in the form of a pulsed flow; however, the fluid may be continuously ejected. When the fluid container 760 is formed of an infusion solution bag that accommodates a fluid, it is possible to perform the fine pressure decrease adjustment control operation as follows. That is, it is possible to perform the fine pressure decrease adjustment control operation by opening the air vent valve 812 for a very small amount of time and decreasing the pressure of the pressurized chamber 800 by a very small amount of pressure.

In the fluid ejection device 1 with the above-mentioned configuration, once the pressure of the fluid container 760 is increased to an amount of pressure required to eject the fluid, the pressure is maintained for approximately one to approximately two hours. In other words, there is a problem in that the pressure of the fluid container 760 decreases when one to two hours have elapsed after the pressure is increased to an amount of pressure required to eject the fluid. Accordingly, after a predetermined amount of time has elapsed after the pressure of the fluid container 760 is increased to an amount of pressure required to eject the fluid, the ejection mode may be reset to the trial mode by turning on the trial flag.

An ejection time in the trial mode required to switch the trial mode to the normal ejection mode may be specified. That is, the ejection mode can be configured such that the ejection mode is not switched to the normal ejection mode until a specified amount of time has elapsed from the ejection of the fluid in the trial mode. In this manner, it is possible to reliably increase the inner pressure of the fluid container 760 to a proper pressure until the ejection mode is switched to the normal ejection mode.

The embodiment is given to help understanding the invention, and the interpretation of the invention is not limited to the embodiment. Modifications or improvements can be made to the invention insofar as the modifications or the improvements do not depart from the spirit of the invention, and the invention includes the equivalent.

What is claimed is:

1. A fluid ejection device comprising:
a fluid ejection unit that ejects a fluid;
an ejection control unit that receives a fluid ejection command input, and controls the ejection of the fluid from the fluid ejection unit;
a fluid container that accommodates the fluid to be supplied to the fluid ejection unit;
a connection channel that connects the fluid ejection unit and the fluid container, and acts as a channel through which the fluid flows;
an opening and closing unit that opens and closes the connection channel;
a volume change unit that controls the opening and closing unit to open and close the connection channel, and adjusts an inner volume of the fluid container; and
a pressure detection unit that detects an inner pressure of the fluid container,
wherein when the fluid ejection device receives the ejection command input, the fluid ejection device is configured to operate
in a first ejection mode in which, regardless of an amount of the inner pressure of the fluid container, in a state where the volume change unit control the opening and closing unit to open the connection channel, the volume change unit reduces the inner volume, and the ejection control unit ejects the fluid from the fluid ejection unit, and after the ejection of the fluid, the volume change unit controls the opening and closing unit to close the connection channel, or
in a second ejection mode in which when the inner pressure of the fluid container is higher than a predetermined pressure, and in a state where the volume change unit controls the opening and closing unit to open the connection channel, the volume change unit reduces the inner volume, and the ejection control unit ejects the fluid from the fluid ejection unit, and after the ejection of the fluid, the volume change unit controls the opening and closing unit to close the connection channel.

2. The fluid ejection device according to claim 1,
wherein when the fluid ejection device receives the ejection command input, the fluid ejection device is configured to operate
in the first ejection mode in which, regardless of an amount of the inner pressure of the fluid container, in a state where the volume change unit controls the opening and closing unit to open the connection channel, the volume change unit reduces the inner volume, and the ejection control unit ejects the fluid from the fluid ejection unit, and after the ejection of the fluid, the volume change unit controls the opening and closing unit to close the connection channel, or
in the second ejection mode in which when the inner pressure of the fluid container is in a predetermined range of pressure, and in a state where the volume change unit controls the opening and closing unit to open the connection channel, the volume change unit reduces the inner volume, and the ejection control unit ejects the fluid from the fluid ejection unit, and after the ejection of the fluid, the volume change unit controls the opening and closing unit to close the connection channel.

3. The fluid ejection device according to claim 1,
wherein the fluid container includes a syringe for accommodating the fluid and a piston, and
wherein the volume change unit changes the inner volume of the fluid container by moving the piston.

4. The fluid ejection device according to claim 3,
wherein in the first ejection mode, the volume change unit moves the piston at a constant speed.

5. The fluid ejection device according to claim 4,
wherein the fluid ejection unit includes a nozzle through which the fluid is ejected, and
wherein in the first ejection mode, the fluid is ejected through the nozzle by a constant speed movement of the piston, and the inner pressure of the fluid container converges to a predetermined pressure.

6. The fluid ejection device according to claim 3,
wherein the piston has a gasket at the tip thereof.

7. A fluid ejection method of a fluid ejection device including a fluid ejection unit that ejects a fluid, an ejection control unit that controls the ejection of the fluid from the fluid ejection unit, a fluid container that accommodates the fluid to be supplied to the fluid ejection unit, a connection channel that connects the fluid ejection unit and the fluid container, and acts as a channel through which the fluid flows, an opening and closing unit that opens and closes the connection channel, a volume change unit that controls the opening and closing unit to open and close the connection channel, and adjusts an inner volume of the fluid container, and a pressure detection unit that detects an inner pressure of the fluid container, the method comprising:
performing a first ejection operation in which when the fluid ejection device receives the ejection command input, regardless of an amount of the inner pressure of the fluid container, the volume change unit reduces the inner volume while controlling the opening and closing unit to open the connection channel, and the volume change unit controls the opening and closing unit to close the connection channel after the ejection control unit ejects the fluid from the fluid ejection unit; and
performing a second ejection operation in which the inner pressure of the fluid container is higher than a predetermined pressure, the volume change unit reduces the inner volume while controlling the opening and closing unit to open the connection channel, and the volume change unit controls the opening and closing unit to close the connection channel after the ejection control unit ejects the fluid from the fluid ejection unit.
